(19) United States
(12) Patent Application Publication
(10) Pub. No.: US 2021/0071355 A1
(43) Pub. Date: Mar. 11, 2021

(54) COMPOSITE FABRIC, METHOD FOR FORMING COMPOSITE FABRIC, AND USE OF A COMPOSITE MATTER FABRIC

(71) Applicant: First Step Holdings, LLC, Tucson, AZ (US)

(72) Inventors: Scott N. SHEFTEL, Tucson, AZ (US); Jeffry B. SKIBA, Chandler, AZ (US); Stanley N. SHEFTEL, Akron, OH (US)

(21) Appl. No.: 16/952,938

(22) Filed: Nov. 19, 2020

Related U.S. Application Data
(63) Continuation-in-part of application No. 15/823,076, filed on Nov. 27, 2017.

(51) Int. Cl. D06M 11/74; D06M 11/83; B29C 48/16; B29C 48/00; D01F 6/04; D01F 1/10; A61N 1/36; A61N 1/04

(52) U.S. Cl. CPC D06M 11/74; D06M 11/83; B29C 48/16; B29C 48/022; D06M 2101/20; D01F 1/10; A61N 1/36003; A61N 1/0496; D01F 6/04

(57) ABSTRACT

A cloth article formed of a thermoplastic or thermosetting material containing particles of metal disbursed there through. More particularly, there is disclosed a fiber material formed of a thermoplastic or thermosetting material containing particles of metal dispersed intermittently within the fiber material during fiber formation, wherein the particles of metal are exposed at least in part on a surface of the fiber material, wherein the fiber material also includes carbon fiber nanotubes added to the fiber material, and wherein the fiber material is woven into a fabric and the fabric is formed into a cloth article.

### CROSS REFERENCE TO RELATED APPLICATIONS

This application is a continuation-in-part of U.S. patent application Ser. No. 15/823,076, filed Nov. 27, 2017, the contents of which are incorporated herein by reference.

### BACKGROUND OF THE INVENTION

Living tissue has inherent electrical nature that includes the creation of voltage, current, capacitance and impedance. The external application of electrical energy to any biological tissue may have therapeutic effects if the delivery method is safe and at an appropriate physiological level. In a human body, electrical charges around a cell may open voltage dependent gates, allowing cellular cytoplasm to contact the extracellular environment. The infinite combinations of voltage, current, capacitance and impedance are employed within living tissue as a foundation of life. However, an understanding of the nature of living state electrical energy is elusive since measurement of energy in the nano and pico volts/ampere range has been confined to a relatively small area of physics. Muscles are activated by electrical action potentials contained within an insulated nerve bundle. External stimuli is converted into electrical impulses stored in the brain and sent down the nerve bundles. In a cellular matrix, the extracellular fluid acts as a conductor and functions independently of the muscle action signals. Afferent and efferent nerves send signals back and forth to the brain in a similar manner, through insulated nerves.

The recent development of smart fabrics that can provide an electrical field over the skin for stimulus, to measure impedance, warm the user and/or provide feedback about the users' health represent novel devices specifically aimed at a physiologic function. By way of example, our earlier U.S. Pat. Nos. 9,192,761 and 9,707,172, the contents of which are incorporated herein by reference, describe methods and devices for treating various conditions including hyperhidrosis and other conditions such as neuropathic pain including peripheral artery disease and neuropathy; surgical rehabilitation and surgical convalescence including joint surgery rehabilitation and soft tissue healing; and physical therapy including muscle and tendon healing and stroke rehabilitation, by applying onto a skin surface of a patient in need of said treatment, a device comprising a fabric or substrate containing elemental zinc particles arranged so that the fabric or substrate forms a plurality of half-cells of an air-zinc battery, whereby to produce an ion exchange with the skin of the patient. Zinc or zinc salt against the skin will result in secondary reactions to form zinc complexes beneficial to the host. The ability to deliver topical zinc to the surface of the skin can have beneficial effects provided the topical zinc is in the correct quantity.

Additionally, the therapeutic value of metals and metal salts such as zinc, zinc oxide and zinc salt in cosmetic and medicinal ointments and creams, i.e., for treating a variety of skin conditions is well documented in the art. However, one of the limitations of creams or ointments is that they require a carrier gel or petrolatum, and these carriers create barriers on the skin, potentially trapping microbes beneath the barriers. Confirmatory studies are required to assure that these creams and ointments are effective in preventing colonization of bacterial strains and resultant biofilms forms of the bacteria, significantly increasing the challenge of any antimicrobial to function.

It has been postulated that many of the same benefits of direct application to the skin of creams or ointments containing zinc may be achieved by bringing a fabric having elemental zinc particles printed thereon, in contact with the skin of the patient, i.e., as described in our aforesaid '761 and '172 patents. However, fabric coated with elemental zinc particles as described above formed by printing zinc particles on the surface of the fabric have limited washability and abrasion resistance. Also, in the case of thermoplastics, once we exceed about 30% solids in the melt, the strength of the fiber drops considerably. There are many thermosetting and thermoplastic polymers as well as other “binders” such as printer's ink, silicone, natural collagen or cellulose binders that could be used to suspend the metal powder (or salt thereof) or combination of metals within the fiber, thread or yarn. However, prior to the present invention, no one has successfully produced metal-filled fabrics having good washability and abrasion resistance.

### OBJECTS OF THE INVENTION

It is therefore an object of the present invention to provide a method for producing metal-filled fabrics, i.e., fabrics having elemental zinc particles or other elemental metal particles, as well as oxides and salts of such metals or combinations of metals with other chemicals carried in or on a fabric, to fabrics so produced, and to methods for treating various conditions using the so produced fabrics.

### SUMMARY OF THE INVENTION

In one aspect the present invention provides method for producing metal particle filled fibers and to metal particle filled fibers produced thereby.

In another and preferred aspect, the metal particles include zinc particles, zinc oxide particles, or zinc salt particles.

In another and preferred aspect, the metal particles have a particle sized range of 1 micron-200 microns, more preferably 2-100 microns, even more preferably 2-10 microns. The metal particles preferably have an average particle size of less than about 10 microns, more preferably less than about 6 microns, even more preferably less than about 5 microns. The reason for these limitations are purely practical since the fiber spinnarettes will plug up if the particles are too large or if they clump together. In addition, if there is too much filler compared to polymer, the fiber will weaken. We could add the reinforcing carbon fiber nanotubes to increase the polymer tensile strength but doing so takes up space in the polymer that we would prefer to fill with the metal.

In still another aspect, the metal particles preferably comprise about 50 and 50%, by volume, of the fiber, more preferably about 40-60 volume % of the fiber, even more preferably between about 20-30 volume % of the fiber.

In yet another aspect of the invention, the metal particles are dispersed as micro pellets within the fiber material.

In yet another aspect, the metal particle filled fiber material is formed by dispersing metal particles throughout the fiber during fiber formation.

In yet another aspect of the invention, the metal particle containing fiber is formed by mixing the metal particles with a thermosetting setting plastic material such as a polyester resin or a vinyl ester resin and forming the mixture as elongate fibers or threads as it sets. Alternatively, the metal particles can be dusted onto the setting fibers or threads.

In yet another aspect of the invention, the metal particle containing fiber is formed by spinning, drawing or extruding a heated thermoplastic material such as a polyolefin such as polyethylene or polypropylene, a polyamide such as nylon, or an acrylic, containing the metal particles.

The amount of metal available per fiber can be manipulated to increase/decrease concentration and spacing of reservoirs of the metal within the fiber. Metal availability also may be controlled by particle size or particle size distribution. Very fine particles may become coated with binder more than larger particles. However, the binder can be manipulated to expose more of the particle to the contact area. By controlling the particle size, performance of the fiber will differ.

The amount of metal available per thread or yarn also can be manipulated to increase/decrease concentration and spacing of reservoirs of the metal within the thread or yarn. This may be done at the fiber level by adjusting the amount of metal held within the fiber and how the metal is attached to the fiber. We can fill the fiber with a large amount or a small amount of metal, or we can co-extrude metal filled fiber over another fiber so the only part of the fiber loaded with metal is the outer wrap. We also can manipulate the extrusion to create pockets of high and low metal concentrations, or no metal at all.

In the case of a monofilament we can “bump extrude” the filament with metal to produce thicker portions metal filled filament and thinner portions created by the frequency of the “bumps”.

By controlling the amount and particle size of metals in the fiber and how the metal is bound to the fiber, we can adjust slow or fast release of ions. We also can increase or decrease the reservoir capacity within the fiber and subsequently the capacity of the battery created when combined with oxygen.

### BRIEF DESCRIPTION OF THE DRAWINGS

Further features and advantages of the present invention will be seen from the following detailed description, taken in conjunction with the accompanying drawings, wherein like numerals depict like parts, and wherein:

FIG. 1 is a flow diagram showing one method for forming a metal particle filled fiber in accordance with the present invention;

FIG. 2 is a flow diagram showing an alternative method for forming a metal particle filled fiber in accordance with the present invention;

FIG. 3 is a side elevational view of a monofilaments fiber made in accordance with the present invention;

FIG. 4 is a side elevational view of a metal particle filled fiber made in accordance with the present invention;

FIG. 5 is a top plan view of a fabric made from a monofilaments fiber of FIG. 3 in accordance with the present invention;

FIG. 6A-6E illustrates patterns of metal deposition on fabric used for making articles of clothing in accordance with the present invention; and

FIG. 7 is a plan view showing various articles of clothing and wraps made in accordance with the present invention.

### DETAILED DESCRIPTION OF THE INVENTION

In the following description, the term “metal particles” may include elemental metal particles of metals capable of forming metal-air electrochemical cells, and oxides and salts thereof. Preferred are zinc metal particles and oxides and salts thereof, although other metals and oxides and salts thereof may be used including aluminum, iron, copper, or magnesium.

The term “fibers” may comprise both natural and synthetic fibers, filaments and threads, although synthetic fibers are preferred, in particular, fibers formed of thermoplastic or thermosetting plastic materials.

As used herein “metal filled fibers” means fibers, having metal particles carried on or within the fibers, and in which the metal particles are at least in part exposed to air.

The present invention provides a method forming metal particle filled fibers suitable for weaving or knitting into cloth for use in treating hyperhidrosis or neuropathy, or other conditions according to our prior '761 and '172 patents, incorporated herein by reference, and other conditions as above discussed. More particularly, the present invention provides a method for producing metal particle containing fibers that are capable of standing up to washing (at least 20 washes) abrasion resistance, and have the ability to release ions when in contact with a patient's skin.

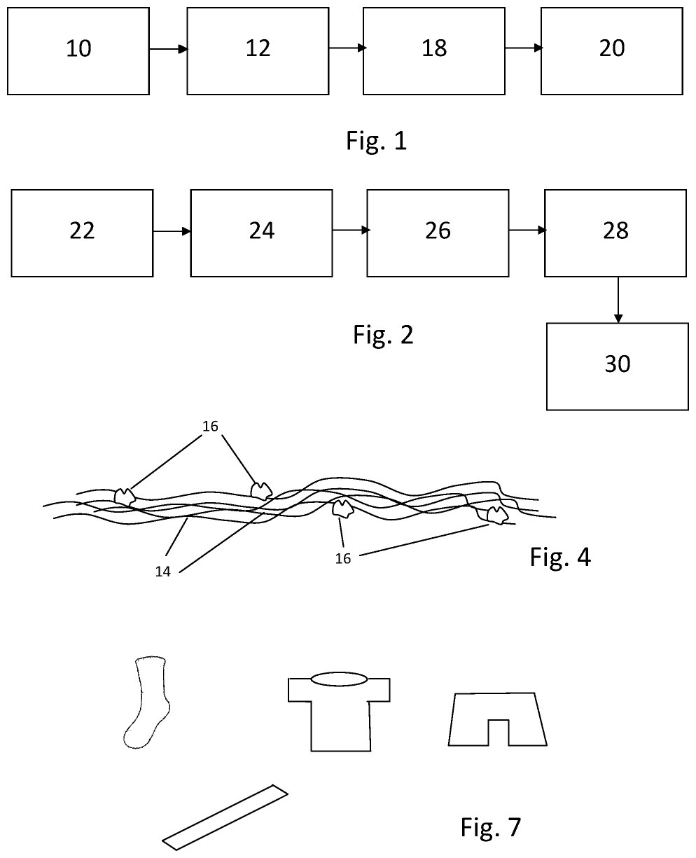

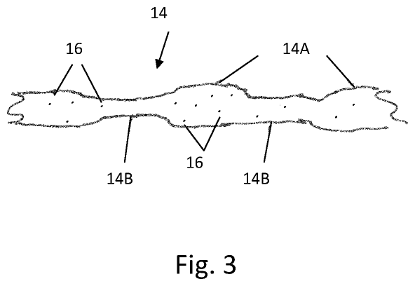

Referring to FIG. 1, according to a first embodiment of our invention, metal particles, typically metallic zinc particles which may be previously formed by grinding or precipitated out of suspension, and having an average particle size between 1 and 100 nanometers, more preferably 1-10 microns, even more preferably about 5 microns are mixed with a thermoplastic material such as polyethylene in a heated mixing vat 10 to melt the material, and the mixture bump extruded or melt spun at spinning station 12 to form fibers 14, having thicker portions 14A of metal particles 16 filled filaments and thinner portions 14B of metal particles 16 filled filaments therebetween (see FIGS. 3 and 4). The polyethylene is the polymer of choice for releasing of electrons from the metal. The porosity of the fiber also is believed to play a part. Polyacrylic or polyester fibers also may be used however the result is a slower ion release. The metal particles filled fibers may then be cabled or twisted at a cabling station 18, and woven at a weaving or knitting station 20 into a garment such gloves, hats, socks, underwear, bras and underbra inserts, shirts, leggings, tights, compression clothing or a cloth which may be made into a therapeutic wrap (see FIG. 7) for use in treating hyperhidrosis, neuropathy and other condition as described in our aforesaid '761 and '172 patents, incorporated herein by reference.

Referring to FIG. 2, according to a second embodiment of the invention, metal particles, typically metallic zinc particles having an average particle size between 1 and 100 microns, preferably 1-10 microns, even more preferably about 5 microns are mixed with a thermosetting polymer material such as polyester chips in a melting vat 22. The molten mixture is expressed through a spinneret at station 24 to form an elongate thread having metal particles incorporated into the thread with the metal particles exposed at least in part on the surface of the thread. Alternatively, pure polyester chips may be spun or pulled from the melt, and dusted with metal particle as the thread sets. The thread is then cabled or twisted at a cabling station 26, woven into cloth at a weaving station 28, and the cloth formed into a textile product or wrap at step 30.

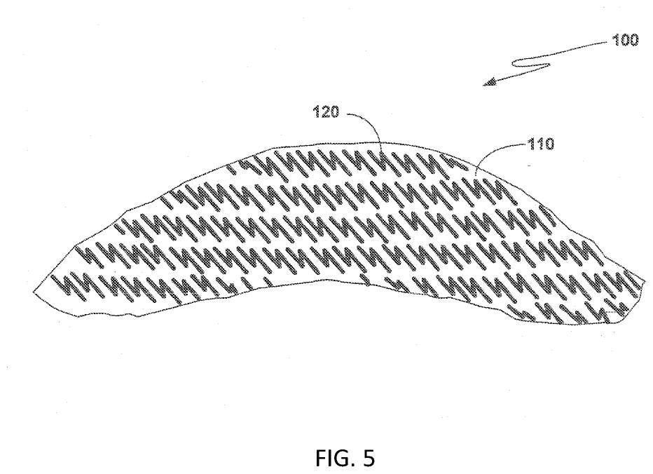

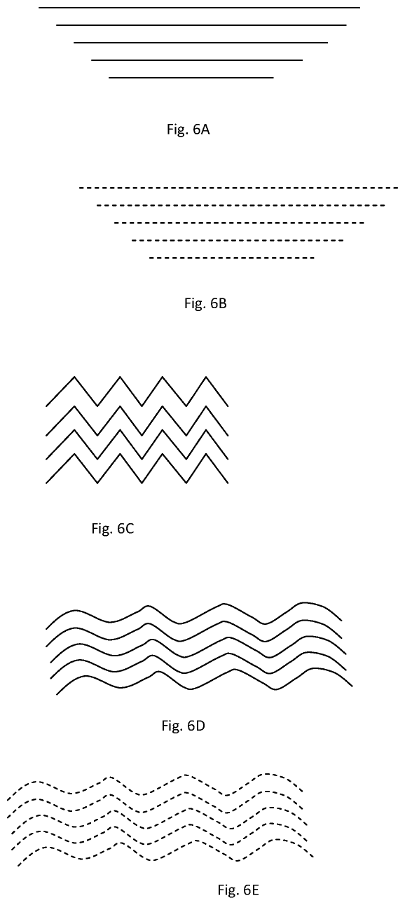

Referring to FIG. 5, an embodiment of Applicants' device for treating hyperhidrosis is illustrated. As shown, Applicants' device comprises an underbra insert 100 that includes a fabric 110 and a plurality of metal deposition areas 120. As shown, the plurality of individual metal deposition areas 120 are discontinuous and uniformly distributed on the surface of the fabric 110, in imaginary spaced lines or lines of dots, to cover a substantially consistent percentage of the surface area of the fabric 110. Typically, the lines or lines of dots are evenly spaced at spacings from 0.1 to 3 mm, preferably 0.2 to 2 mm, more preferably 0.3 to 1.5 mm, most preferably 0.5 to 1.0 mm. The concentration of zinc in the binder that forms the line or deposition determines the amount of zinc available for the battery. Preferred concentration is 30% but the lowest is about 1% and the highest about 50%. The mixture of binder and metal forms a paste that can be applied by silk screening wherein the paste viscosity is important. A 30% by weight zinc to binder is preferred for this. The line width and length also determines the amount of zinc in the deposition since the wider and longer the line, the more zinc is available. Preferred line or line of dots width is 1 mm width but width can vary from 0.1 mm up to 5 mm width. Since the deposition is on a fabric, the amount of binder/zinc applied also can be varied. In certain embodiments, the article being coated can be coated twice or more times over the same spot wherein the thickness of the deposition can be increased as desired. In certain embodiments, the metal deposition area patterns 120 cover from about 10% to about 90% of the surface area of the fabric. In other embodiments, the metal deposition areas 120 cover from about 20% to about 80%, from about 15% to about 75%, from about 25% to about 50%, or from about 30% to about 40% of the surface area of the fabric 110. Although FIG. 5 shows the plurality of metal deposition areas 120 substantially uniformly distributed on the surface of the fabric 110, in other embodiments, the plurality of metal deposition areas 120 randomly may be distributed on the surface of the fabric 110. Typically, the lines have a thickness of 0.1 to 3 mm, preferably 0.2 to 2 mm, more preferably 0.3 to 1.0, most preferably 0.4 to 0.5 mm. The spaced lines may be continuous and may take various forms including straight, curved and various angular shapes as shown, for example, straight continuous lines are shown in FIG. 6A; straight broken lines are shown in FIG. 6B; continuous saw-shaped as shown in FIG. 6C; continuous wavy lines as shown in FIG. 6D; broken wavy lines as shown in FIG. 6E, etc. The actual shape of the lines is not important. Preferably, but not necessarily, the lines are approximately equal in thickness and are evenly spaced.

The underbra insert fabric 110, as illustrated in the embodiment of FIG. 5, comprises a single layer. However, in other embodiments, the fabric 110 may comprise one, two, or three or more layers of fabric including metal deposition areas on at least one surface of the device. The underbra insert 100 is worn inside a bra cup underneath the breast in contact with the skin as a bra underliner to treat excessive sweating associated with hyperhidrosis.

Preferably, but not necessarily, the fabric 110 comprises a woven textile, a non-woven textile, a fibrous mesh, a non-fibrous mesh, a textile mesh, or the like. In one embodiment, the fabric may comprise a polymeric film or a polymeric coating. In an embodiment, the fabric may be interwoven with elastic fibers, elastic bands, or metallic fibers. In certain embodiments, the fabric is electrically conductive or electrically non-conductive.

In certain embodiments, fabric 110 is permeable to ambient air. In certain embodiments, the plurality of individual metal deposition areas 120 comprise elemental zinc particles.

In one embodiment, the device includes a fastener configured to attach the device or the underbra insert 100 to the skin surface or to the surface of a cloth article. For example, referring again to FIG. 5, in certain embodiments the surface of the fabric 110 comprises a surface of the fabric 110 including the plurality of metal deposition areas 120 in contact with the skin and an opposing surface of the fabric 110 in contact with an a cloth article. In certain embodiments, the opposing surface of the fabric 110 includes an adhesive configured to attach the fabric 110 to a cloth article. For example, the underbra insert 100 as shown in FIG. 1 includes the plurality of metal deposition areas 120 on one surface of the fabric 110 configured for contact with the skin surface. An opposite surface of the underbra insert 100 (not shown) includes an adhesive or adhesive strips configured to adhere the underbra insert 100 to the interior of a bra surface. In an embodiment, the device is configured for attachment to a cloth article via at least one of the group consisting of a VELCRO® fastener, buttons, zippers, electrostatics, an adhesive, a hook and eye fastener, a thread, snaps, or the like.

In an embodiment, the surface of the fabric 110 including the plurality of metal deposition areas 120 further comprises an adhesive for attachment of the fabric to the skin surface. In an embodiment, the fabric of the device is flexible and/or conformable to the skin surface. In certain embodiments, the fabric of the device is compressive to the skin surface, for example and without limitation, a sock, a glove, a headband, or an elastic bandage or wrap.

In an embodiment, the fabric of the device comprises a cloth article. For example, the fabric includes at least one member selected from the group consisting of a sock, a glove, a scarf, a headband, a cap, a hat, a face mask, a respirator, a t-shirt, a bra, an underarm or underbra insert, pants, sleeves, underwear (undergarment clothing in contact with the skin), or compression clothing such as ankle, arm or knee sleeves, shorts and shirts, or sheets and pillowcases, towels and drapes.

In certain embodiments, zinc is utilized as a powdered elemental crystal. In certain embodiments, the zinc utilized has a purity of about 99.99 percent however, zinc is available in other purities and particle sizes as defined by the user. In certain embodiments, the zinc comprises a −325 mesh size. As those skilled in the art will appreciate, particles passing through a −325 mesh are considered the “fines.”

In certain embodiments, the zinc particles are very uniform in size. In certain embodiments, the zinc particle size distribution is between about 4 microns to about 10 microns in diameter. These individual particle crystals approach the visible range and are easily seen as shiny crystals on the surface.

In certain embodiments, Applicants' socks comprise a woven fabric. In certain embodiments, Applicants' cloth articles are formed of a non-woven fabric. In certain embodiments, Applicants' cloth articles are formed of a braided fabric. In certain embodiments, Applicants' cloth articles comprise a polymeric fabric. In certain embodiments, Applicants' cloth articles are permeable to ambient oxygen.

The present invention is unique in that the zinc pattern grid creates a matrix of individual half-cells (anodes) for ion exchange with the skin. One-half cell of electrochemical reaction is the zinc impregnated fabric (the anode), and the other is the skin of the human or animal, supplying moisture and oxygen (the cathode) completing the circuit for microcurrent production. Alternatively, the oxygen may be supplied, in part, from ambient air.

The chemistry of Zinc-air batteries is instructive. Such batteries are powered by oxidizing zinc with oxygen from the air. During discharge, zinc particles form a porous anode, which is saturated with an electrolyte, namely sweat. Oxygen from the air reacts at the cathode and forms hydroxyl ions which migrate into the zinc paste and form zinc hydroxide Zn(OH)₂, releasing electrons to travel to the cathode.

The chemical equations for the zinc-air battery formed using Applicants' zinc-coated socks and ambient oxygen include:

Anode: Zn+4OH⁻→Zn(OH)₄²⁻+2e⁻(E₀=−1.25 V)

Fluid: Zn(OH)₄²⁻→ZnO+H₂O+2OH⁻

Cathode: ½O₂+H₂O+2e⁻→2OH⁻(E₀=0.34 V)

Overall, the zinc oxygen redox chemistry recited immediately hereinabove comprises an overall standard electrode potential of about 1.59 Volts.

There is a certain amount of gas exchange at the skin surface with a partial pressure of oxygen. The oxygen at the skin surface is a product of ambient oxygen in addition to oxygen diffusion from capillary blood flow. In certain embodiments, the zinc in contact with a patient's skin resulting from wearing, for example, our zinc-containing socks, in combination with sweat and transcutaneous oxygen complete the galvanic circuit described hereinabove.

The chemistry utilized by Applicants' zinc-coated cloth articles socks differs from a more conventional galvanic cell. A galvanic cell, or voltaic cell is an electrochemical cell that derives electrical energy from spontaneous redox reactions taking place within the cell. It generally consists of two different metals connected by a salt bridge, or individual half-cells separated by a porous membrane. In contrast, the chemistry of Applicants' zinc-air battery does not require use of a second metal. Applicants' method to treat hyperhidrosis utilizes elemental zinc particles disposed onto a fabric, where the elemental zinc particles are in contact with the skin. In certain embodiments, other than elemental zinc metal and zinc oxides formed therefrom, no other or additional metals or metal oxides are needed or are utilized in Applicants' method and device.

In certain embodiments, a method for treating hyperhidrosis includes disposing onto a skin surface a device including a fabric having elemental zinc particles disposed thereon. The fabric is configured to contact the skin and to generate an electric current and metal ions when oxidized by ambient oxygen. The generation of such an electric current results in reducing the amount of sweat disposed on the skin surface thereby providing a treatment for hyperhidrosis.

In certain embodiments, Applicants' method for treating hyperhidrosis includes generating an electric current on the skin surface resulting in a reduction of an amount of sweat released by the skin. For example, in a non-limiting embodiment, the method includes contacting a skin surface with elemental zinc particles disposed on at least a portion of the fabric or flexible substrate.

The method described herein may include any of the fabric and metal materials previously described with respect to the exemplary device described herein, FIG. 7 shows various examples of clothing items and wraps made in accordance with the present invention including socks, gloves, hats, underwear, bra, t-shirts, leggings and tights, wraps, compression clothing, etc.

Various changes may be made in the above invention without departing from the spirit and scope. For example, the fibers may be co-extruded to have a center or core of the same or dissimilar polymer with the metal filled polymer on the outside of the fiber. Or, the metal filled polymer may be intermittently dispersed into discrete reservoirs within the fiber during fiber formation. And, we can overcome prior art limitations of fiber manufacturing with the addition of carbon fiber nanotubes (hollow-tubes) that can provide increased tensile strength as well as the antimicrobial nature of the hollow tubes. In addition we can add prior to fiber manufacturing additives such as carbon fiber nanotubes carrying drugs to target specific cells within the host. These fibers, once spun into threads or yarns and manufactured in to a fabric will contact the target tissue closely. Also, the amount of metal particles in the fibers may be adjusted to adjust the capacity or voltage of the air battery in the thread or yarn.

1. A cloth article formed of a reinforced fabric material formed of polyethylene fibers containing particles of metal and carbon fiber nanotubes dispersed intermittently within the polyethylene fibers during fiber formation, wherein the particles of metal are selected from the group consisting of elemental zinc particles, zinc oxide particles, wherein the particles have a size range of 1-200 microns, and wherein the particles comprise between 40-60 volume % of the polyethylene fibers, and are exposed at least in part on a surface of the polyethylene fibers, wherein the reinforced fabric material is formed by co-extruding polyethylene fibers with a core fiber formed of a different thermoplastic material or with a thermosetting material, wherein the polyethylene fibers contain particles of metal exposed on a surface of the polyethylene fibers wherein the cloth article is configured to be in direct contact with the skin of a user, at least in part, wherein the particles are arranged so that the fabric in contact with the skin of the wearer forms a plurality of half-calls of an air-zinc battery, and wherein the cloth article is selected from a group consisting of socks, gloves, headbands, caps, scarves, face masks, respirators, hats, t-shirts, leggings, tights, underwear, underarm and under bra inserts bras, and compression clothing and elastic bandages and wraps, sheets and pillowcases, towels and drapes, in which the particles of metal exposed at least in part on the surface of the polyethylene fibers contact the skin of the user.

2. The cloth article of claim 1, wherein the particles of metal have a particle size range of 1-100 microns.

3. The cloth article of claim 1, wherein the reinforced fabric material comprises polyethylene fiber sections containing the particles of metal and polyethylene fiber sections devoid of particles of metal.

4. The cloth article of claim 1, wherein the reinforced fabric material further includes a drug carried by/on the carbon fiber nanotubes.

5. The cloth article of claim 1, wherein the particles of metal have a particle size range of 2-100 microns.

6. The cloth article of claim 1, wherein the particles of metal have a particle size range of 2-10 microns.

7. The cloth article of claim 2, wherein the particles of metal have a particle size range of 1-10 microns.

8. The cloth article of claim 2, wherein the particles of metal have a particle size range of 5-6 microns.

9. The cloth article of claim 1, wherein the zinc particles are arranged in a plurality of evenly spaced lines.
